(19) United States
(12) Patent Application Publication
(10) Pub. No.: US 2018/0340028 A1
(43) Pub. Date: Nov. 29, 2018

(54) METHODS FOR THE TREATMENT OF EPILEPSY

(71) Applicant: Genentech, Inc., South San Francisco, CA (US)

(72) Inventor: Sharmila Rajan, South San Francisco, CA (US)

(73) Assignee: Genentech, Inc., South San Francisco, CA (US)

(21) Appl. No.: 15/926,149

(22) Filed: Mar. 20, 2018

Related U.S. Application Data
(63) Continuation of application No. PCT/US2016/053506, filed on Sep. 23, 2016.
(60) Provisional application No. 62/222,983, filed on Sep. 24, 2015.

(51) Int. Cl. C07K 16/28; A61P 25/08

(52) U.S. Cl. CPC C07K 16/2863; A61P 25/08; A61K 2039/505; C07K 2317/34; C07K 2317/75; C07K 2317/31

(57) ABSTRACT

The invention provides methods for the treatment of seizures and epilepsy using FGF21 receptor activators.

### CROSS REFERENCE TO RELATED APPLICATIONS

This application is a Continuation of International Application No. PCT/US2016/053506, filed Sep. 23, 2016 which claims benefit of priority under 35 U.S.C 119 to U.S. provisional application Ser. No. 62/222,983, filed Sep. 24, 2015. Each of which are incorporated herein by reference in its entirety.

### FIELD OF THE INVENTION

The present invention relates to methods for the treatment of seizures and epilepsy using FGF21 receptor activators.

### SEQUENCE LISTING

The instant application contains a Sequence Listing submitted via EFS-Web and hereby incorporated by reference in its entirety. Said ASCII copy, created on Mar. 9, 2018, is named Sequence Listing.txt and is 16,819 bytes in size.

### BACKGROUND

Epilepsy is a condition in which a person has recurrent seizures due to a chronic, underlying process. Up to 1% of the individuals have epilepsy and in the United States approximately 2.5 million individuals have epilepsy and approximately one quarter of them have inadequately controlled seizures under current therapy adequately controls seizures.

There is no completely effective therapy for epilepsy, but there are several approaches currently used for patient treatment. There are a number of antiepileptic drugs that have been approved, but the response rates are less than 50% for any particular drug. In addition, certain patients are eligible for surgery, which provides substantial improvement in that subpopulation. Finally, there is evidence that a ketogenic diet may be therapeutically useful, especially in pediatrics patients, but the diet is a difficult one to which to adhere (see, e.g., Neal et al, “The ketogenic diet for the treatment of childhood epilepsy: a randomized controlled trial.” Lancet Neurol. 7: 500-06 (2008)). Accordingly, it remains of great interest to identify additional possible therapeutic options for individuals with epilepsy.

### SUMMARY

The invention provides methods for the treatment of seizures and epilepsy using FGF21 receptor activators.

In one aspect, the invention provides the use of an FGF21 receptor activator in the manufacture of a medicament for the treatment of epilepsy. In some embodiments, the FGF21 receptor activator is selected from the group consisting of FGF21, an anti-FGFR1c antibody, an anti-KLB antibody, and a bispecific anti-FGFR1c/KLB antibody. In some embodiments, the FGF21 receptor activator is FGF21. In some embodiments, the FGF21 is conjugated to a heterologous molecule. In some embodiments, the heterologous molecule is PEG. In some embodiments, the heterologous molecule is a polypeptide, e.g., an antibody Fc. (e.g. from IgG1 antibody).

In some embodiments, the FGF21 receptor activator is an anti-FGFR1c antibody. In some embodiments, the anti-FGFR1c antibody binds to a peptide selected from the group consisting of KLHAVPAAKTVKFKCP (SEQ ID NO: 3) and FKPDHRIGGYKVRY (SEQ ID NO: 4).

In some embodiments, the FGF21 receptor activator is an anti-KLB antibody. In some embodiments, the anti-KLB antibody is wherein the anti-KLB antibody is selected from the group consisting of 16H7 (as described in US 2011/0135657) and h5h23 (described in US 2015/0210764), or derivatives thereof. In this context, a “derivative” of an antibody is one which has one or more amino acid insertions, deletions or substitutions and still binds to and KLB and activates the FGF21 receptor.

In some embodiments, the FGF21 receptor activator is a bispecific anti-FGFR1c/KLB antibody. In some embodiments, the bispecific anti-FGFR1c/KLB antibody binds to a KLB epitope within a fragment of KLB consisting of the amino acid sequence SSPTRLAVIPWGVRKLLRWVRRNYGDMDIYITAS (SEQ ID NO: 5). In some embodiments, the bispecific anti-FGFR1c/KLB antibody comprises an anti-FGFR1c arm comprising amino acid sequences from YW182.5 YGDY and an anti-KLB arm comprising amino acid sequences from anti-8C5.K4.M4L.H3.KNV (as described in US 2015/0218276).

In some embodiments, the medicament is administered subcutaneously. In some embodiments, the medicament is for administration with one or more additional therapeutics selected from the group consisting of: levetiracetam (“KEPPRA™”), Levetiracetam Extended Release (XR) (“KEPPRA XR™”) lamotrigine (“LAMICTAL™”), lamotrigine XR (“LAMICTAL XR™”), oxycarbazepine (“TRILEPTAL®”), carbamazepine (“TEGRETOL®”), lacosamide (“VIMPAT®”), valproic acid (“WA”), and perampanel (“FYCOMPA®”).

In one aspect, the invention provides a method of treating epilepsy in an individual comprising administering to the individual an effective amount of an FGF21 receptor activator. In some embodiments, the FGF21 receptor activator is selected from the group consisting of FGF21, an anti-FGFR1c antibody, an anti-KLB antibody, and a bispecific anti-FGFR1c/KLB antibody. In some embodiments, the FGF21 receptor activator is FGF21. In some embodiments, the FGF21 is conjugated to a heterologous molecule. In some embodiments, the heterologous molecule is PEG. In some embodiments, the heterologous molecule is a polypeptide, e.g., an antibody Fc. (e.g. from IgG1 antibody).

In some embodiments, the FGF21 receptor activator is an anti-FGFR1c antibody. In some embodiments, the anti-FGFR1c antibody binds to a peptide selected from the group consisting of KLHAVPAAKTVKFKCP (SEQ ID NO: 3) and FKPDHRIGGYKVRY (SEQ ID NO: 4).

In some embodiments, the FGF21 receptor activator is an anti-KLB antibody. In some embodiments, the anti-KLB antibody is wherein the anti-KLB antibody is selected from the group consisting of 16H7 (as described in US 2011/0135657) and h5h23 (described in US 2015/0210764), or derivatives thereof. In this context, a “derivative” of an antibody is one which has one or more amino acid insertions, deletions or substitutions and still binds to and KLB and activates the FGF21 receptor.

In some embodiments, the FGF21 receptor activator is a bispecific anti-FGFR1c/KLB antibody. In some embodiments, the bispecific anti-FGFR1c/KLB antibody binds to a KLB epitope within a fragment of KLB consisting of the amino acid sequence SSPTRLAVIPWGVRKLLRWVRRNYGDMDIYITAS (SEQ ID NO: 5). In some embodiments, the bispecific anti-FGFR1c/KLB antibody comprises an anti-FGFR1c arm comprising amino acid sequences from YW182.5 YGDY and an anti-KLB arm comprising amino acid sequences from anti-8C5.K4.M4L.H3.KNV (as described in US 2015/0218276).

In some embodiments, the FGF21 receptor activator is administered subcutaneously. In some embodiments, the method further comprises administering one or more additional therapeutics selected from the group consisting of: levetiracetam (“KEPPRA™”), Levetiracetam Extended Release (XR) (“KEPPRA XR™”), lamotrigine (“LAMICTAL™”), lamotrigine XR (“LAMICTAL XR™”) oxycarbazepine (“TRILEPTAL®”), carbamazepine (“TEGRETOL®”), lacosamide (“VIMPAT®”), valproic acid (“VPA”), and perampanel (“FYCOMPA®”).

### DETAILED DESCRIPTION OF EMBODIMENTS OF THE INVENTION

### I. Definitions

The term “epilepsy,” as used herein, refers to a clinical phenomemon where an individual has two or more unprovoked seizures. Epilepsy includes, e.g., generalized-onset seizures and focal-onset seizures (symptomatic and idiopathic), including childhood absence epilepsy, juvenile myoclonic epilepsy, epilepsy with grand-mal seizures upon awakening, temporal lobe epilepsy, frontal lobe epilepsy, parietal lobe epilepsy, occipital lobe epilepsy, and epileptic encephalopathies, including Ohtahara syndrome, West syndrome, Dravet syndrome, epilepsy with myoclonic atonic seizures, and Lennox-Gastaut syndrome.

The term “FGFR1c,” as used herein, refers to any native fibroblast growth factor receptor 1c (FGFR1c) from any vertebrate source, including mammals such as primates (e.g. humans) and rodents (e.g., mice and rats), unless otherwise indicated. The term encompasses “full-length,” unprocessed FGFR1c as well as any form of FGFR1c those results from processing in the cell. The term also encompasses naturally occurring variants of FGFR1c, e.g., splice variants or allelic variants. The amino acid sequence of an exemplary human FGFR1c is:

(SEQ ID NO: 1)
MWSWKCLLFWAVLVTATLCTARPSPTLPEQAQPWGAPVEVESFLVHPGDL
LQLRCRLRDDVQSINWLRDGVQLAESNRTRITGEEVEVQDSVPADSGLYA
CVTSSPSGSDTTYFSVNVSDALPSSEDDDDDDDSSSEEKETDNTKPNPVA
PYWTSPEKMEKKLHAVPAAKTVKFKCPSSGTPNPTLRWLKNGKEFKPDRI
GGYKVRYATWSIIMDSVVPSDKGNYTCIVENEYGSINHTYQLDVVERSPH
RPILQAGLPANKTVALGSNVEFMCKVYSDPQPHIQWLKHIEVNGSKIGPD
NLPYVQILKTAGYNTTDKEMEVLHLRNVSFEDAGEYTCLAGNSIGLSHHS
AWLTVLEALEERPAVIVITSPLYLEIIIYCTGAFLISCMVGSVIVYKMKS
GTKKSDFHSQMAVEIKLAKSIPLRRQVTVSADSSASMNSGVLLVRPSRLS
SSGTPMLAGVSEYELPEDPRWELPRDRLVLGKPLGEGCFGQVVLAEAIGL
DKDKPNRVTKVAVKMLKSDATEKDLSDLISEMEMMKMIGKEIKNIINLLG
ACTQDGPLYVIVEYASKGNLREYLQARRPPGLEYCYNPSHNPEEQLSSKD
LVSCAYQVARGMEYLASKKCIHRDLAARNVLVTEDNVMKIADFGLARDIH
HIDYYKKTTNGRLPVKWMAPEALFDRIYTHQSDVWSFGVLLWEIFTLGGS
PYPGVPVEELFKLLKEGHRMDKPSNCTNELYMMMRDCWHAVPSQRPTFKQ
LVEDLDRIVALTSNQEYLDLSMPLDQYSPSFPDTRSSTCSSGEDSVFSHE
PLPEEPCLPRHPAQLANGGLKRR.

The terms “anti-FGFR1c antibody” and “an antibody that binds to FGFR1c” refer to an antibody that is capable of binding FGFR1c with sufficient affinity such that the antibody is useful as a diagnostic and/or therapeutic agent in targeting FGFR1c. In one embodiment, the extent of binding of an anti-FGHUc antibody to an unrelated, non-FGFR1c protein is less than about 10% of the binding of the antibody to FGFR1c as measured, e.g., by a radioimmunoassay (RIA). In certain embodiments, an antibody that binds to FGFR1c has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹M to 10⁻¹³M). In certain embodiments, an anti-FGFR1c antibody binds to an epitope of FGFR1c that is conserved among FGFR1c from different species.

The term “KLB,” as used herein, refers to any native klotho beta (KLB) from any vertebrate source, including mammals such as primates (e.g. humans) and rodents (e.g., mice and rats), unless otherwise indicated. The term encompasses “full-length,” unprocessed KLB as well as any form of KLB that results from processing in the cell. The term also encompasses naturally occurring variants of KLB, e.g., splice variants or allelic variants. The amino acid sequence of an exemplary human KLB is:

(SEQ ID NO: 2)
FSGDGRAIWSKNPNFTPVNESQLFLYDTFPKNFFWGIGTGALQVEGSWKK
DGKGPSIWDEIFIHTEILKNVSSTNGSSDSYIFLEKDLSALDFIGVSFYQ
FSISWPRLFPDGIVTVANAKGLQYYSTLLDALVLRNIEPIVTLYHWDLPL
ALQEKYGGWKNDTIIDIFNDYATYCFQMFGDRVKYWITIEINPYLVAWHG
YGTGMHAPGEKGNLAAVYTVGHNLIKAHSKVWEINYNTEIFRPHQKGWLS
ITLGSHWIEPNRSENTMDIFKCQQSMVSVLGWFANPIHGDGDYPEGMRKK
LFSVLPIFSEAEKEIEMRGTADFFAFSFGPNNFKPLNTMAKMGQNVSLNL
REALNWIKLEYNNPRILIAENGWFTDSRVKTEDTTAIYMMKNFLSQVLQA
IRLDEIRVFGYTAWSLLDGFEWQDAYTIRRGLFYVDFNSKQKERKPKSSA
HYYKQIIRENGFSLKESTPDVQGQFPCDFSWGVTESVLKPESVASSPQFS
DPEILYVWNATGNRLLEIRVEGVRLKTRPAQCTDFVNIKKQLEMLARMKV
THYRFALDWASVLPTGNLSAVNRQALRYYRCVVSEGLKLGISAMVTLYYP
THAEILGLPEPLLHADGWLNPSTAEAFQAYAGLCFQELGDLVKLWITINE
PNRLSDIYNRSGNDTYGAAEINLLVAHALAWRLYDRQFRPSQRGAVSLSL
HADWAEPANPYADSHWRAAERFLQFEIAWFAEPLFKTGDYPAAMREYIAS
KEIRRGLSSSALPRLTEAERRLLKGTVDFCALNEIFTTRFVMHEQLAGSR
YDSDRDIQFLQDITRLSSPTRLAVIPWGVRKLLRWVRRNYGDMDIYITAS
GIDDQALEDDRLRKYYLGKYLQEVLKAYLIDKVRIKGYYAFKLAEEKSKP
RFGFFTSDFKAKSSIQFYNKVISSRGFPFENSSSRCSQTQENTECTVCLF
LVQKKPLIFLGCCFFSTLVLLLSIAIFQRQKRRKFWKAKNLQHIPLKKGK
RVVS.

The terms “anti-KLB antibody” and “an antibody that binds to KLB” refer to an antibody that is capable of binding KLB with sufficient affinity such that the antibody is useful as a diagnostic and/or therapeutic agent in targeting KLB. In one embodiment, the extent of binding of an anti-KLB antibody to an unrelated, non-KLB protein is less than about 10% of the binding of the antibody to KLB as measured, e.g., by a radioimmunoassay (RIA). In certain embodiments, an antibody that binds to KLB has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³ M, e.g., from 10⁻⁹M to 10⁻¹³ M). In certain embodiments, an anti-KLB antibody binds to an epitope of KLB that is conserved among KLB from different species.

The term “FGF21 receptor,” as used herein, refers to the receptor complex comprising FGFR1cc and KLB which binds to FGF21.

The term “FGF21 receptor activator,” as used herein, refers to a molecule that activates signaling via the FGF21 receptor. Exemplary FGF21 receptor activators include, e.g., FGF21, optionally conjugated to another molecule, e.g. PEG or the Fc region of an antibody, certain anti-FGFR1c antibodies (described in, e.g., WO 2012/158704), certain anti-KLB antibodies (described in, e.g., US Patent Publications US 2011/0135657, US 2012/0328616, US 2013/0129725, US 2015/0210764), and certain proteins that bind to both FGFR1c and KLB, e.g. non-antibody proteins described in U.S. Pat. No. 8,372,952 and bispecific anti-FGFR1c/anti-KLB antibodies (described in, e.g., US 2015/0218276).

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation or therapeutic molecule, refers to an amount effective, at dosages and for periods of time necessary, to achieve the desired therapeutic or prophylactic result.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The term “package insert” is used to refer to instructions customarily included in commercial packages of therapeutic products, that contain information about the indications, usage, dosage, administration, combination therapy, contraindications and/or warnings concerning the use of such therapeutic products.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment of epilepsy include, but are not limited to, reducing occurrence or recurrence of seizures, alleviation of symptoms, diminishment of any direct or indirect pathological consequences of the disease, decreasing the rate of disease progression, amelioration or palliation of the disease state, or improved prognosis.

### II. Compositions and Methods

In one aspect, the invention is based, in part, on the observation that FGF21 receptor activators demonstrate efficacy in animal models of epilepsy. Accordingly, methods are provided for the treatment of an individual with epilepsy by administering agents that activate the FGF21 receptor.

In some embodiments of the invention, the therapeutic agent is an FGF21 receptor activator. In some embodiments, the FGF21 receptor activator is FGF21 itself, optionally conjugated to another molecule, e.g. PEG or the Fc region of an antibody. In some embodiments, the FGF21 receptor activator is an anti-FGFR1c antibody (see, e.g., antibodies described in WO 2012/158704). In some embodiments, the FGF21 receptor activator is an anti-KLB antibody (see, e.g., US Patent Publications US 2011/0135657, US 2012/0328616, US 2013/0129725, US 2015/0210764). In some embodiments the FGF21 receptor activator is a non-antibody protein that binds to both FGFR1c and KLB (see, e.g. U.S. Pat. No. 8,372,952). In some embodiments, the FGF21 receptor activator is a bispecific anti-FGFR1c/anti-KLB antibody (see, e.g., antibodies described in US 2015/0218276).

Screening for FGF21 receptor activators can be accomplished using methods well known in the art. For example, cells engineered to express the FGF21 receptor complex can be exposed to a candidate activator and any resulting expression and/or phosphorylation states of one or more downstream targets of the FGF21 receptor complex (e.g. ERK) can be analyzed.

Pharmaceutical formulations of an FGF21 receptor activator as described herein are prepared by mixing the FGF21 receptor activator having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized FGF21 receptor activator formulations are described in U.S. Pat. No. 6,267,958. Aqueous FGF21 receptor activator formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide one or more of levetiracetam (“KEPPRA™”), Levetiracetam Extended Release (XR) (“KEPPRA XR™”), lamotrigine (“LAMICTAL™”), lamotrigine XR (“LAMICTAL XR™”), oxycarbazepine (“TRILEPTAL®”), carbamazepine (“TEGRETOL®”), lacosamide (“VIMPAT®”), valproic acid (“VPA”), and perampanel (“FYCOMPA®”). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

In one aspect, an FGF21 receptor activator for use as a medicament is provided. In further aspects, an FGF21 receptor activator for use in treating epilepsy is provided. In certain embodiments, an FGF21 receptor activator for use in a method of treatment is provided. In certain embodiments, the invention provides an FGF21 receptor activator for use in a method of treating an individual having epilepsy comprising administering to the individual an effective amount of the FGF21 receptor activator. In one such embodiment, the method further comprises administering to the individual an effective amount of at least one additional therapeutic agent, e.g., as described below. An “individual” according to any of the above embodiments is preferably a human.

In a further aspect, the invention provides for the use of an FGF21 receptor activator in the manufacture or preparation of a medicament. In one embodiment, the medicament is for treatment of epilepsy. In a further embodiment, the medicament is for use in a method of treating epilepsy comprising administering to an individual having epilepsy an effective amount of the medicament. In one such embodiment, the method further comprises administering to the individual an effective amount of at least one additional therapeutic agent, e.g., as described below. An “individual” according to any of the above embodiments may be a human.

In a further aspect, the invention provides a method for treating epilepsy. In one embodiment, the method comprises administering to an individual having such epilepsy an effective amount of an FGF21 receptor activator. In one such embodiment, the method further comprises administering to the individual an effective amount of at least one additional therapeutic agent, as described below. An “individual” according to any of the above embodiments may be a human.

Such combination therapies noted above encompass combined administration (where two or more therapeutic agents are included in the same or separate formulations), and separate administration, in which case, administration of the FGF21 receptor activator can occur prior to, simultaneously, and/or following, administration of the additional therapeutic agent or agents. In one embodiment, administration of the FGF21 receptor activator and administration of an additional therapeutic agent occur within about one month, or within about one, two or three weeks, or within about one, two, three, four, five, or six days, of each other.

According to the invention, an FGF21 receptor agonist (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

An FGF21 receptor activator would be formulated, dosed, and administered in a fashion consistent with good medical practice. Factors for consideration in this context include the particular disorder being treated, the particular animal being treated, the clinical condition of the individual patient, the cause of the disorder, the site of delivery of the agent, the method of administration, the scheduling of administration, and other factors known to medical practitioners. The FGF21 receptor activator need not be, but is optionally, formulated with one or more agents currently used to prevent or treat the disorder in question. The effective amount of such other agents depends on the amount of FGF21 receptor activator present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of epilepsy, the appropriate dosage of an FGF21 receptor activator (when used alone or in combination with one or more other additional therapeutic agents) will depend on the type of disease to be treated, the type of FGF21 receptor activator, the severity and course of the disease, whether the FGF21 receptor activator is administered for preventive or therapeutic purposes, previous therapy, the patient's clinical history and response to the FGF21 receptor activator, and the discretion of the attending physician. The FGF21 receptor activator is suitably administered to the patient at one time or over a series of treatments. Depending on the type and severity of the disease, about 1 mg/kg to 15 mg/kg (e.g. 0.1 mg/kg-10 mg/kg) of FGF21 receptor activator can be an initial candidate dosage for administration to the patient, whether, for example, by one or more separate administrations, or by continuous infusion. One typical daily dosage might range from about 1 mg/kg to 100 mg/kg or more, depending on the factors mentioned above. For repeated administrations over several days or longer, depending on the condition, the treatment would generally be sustained until a desired suppression of disease symptoms occurs. One exemplary dosage of the FGF21 receptor activator would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an FGF21 receptor activator. The label or package insert indicates that the composition is used for treating the condition of choice. Moreover, the article of manufacture may comprise (a) a first container with a composition contained therein, wherein the composition comprises an FGF21 receptor activator; and (b) a second container with a composition contained therein, wherein the composition comprises a further therapeutic agent. The article of manufacture in this embodiment of the invention may further comprise a package insert indicating that the compositions can be used to treat epilepsy. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### III. Examples

The following are examples of methods and compositions of the invention. It is understood that various other embodiments may be practiced, given the general description provided above.

### Example 1. An Anti-FGFR1c Agonist Antibody Inhibits Seizures in the MES Model

The IVIES is a model for generalized tonic-clonic seizures and provides an indication of a compound's ability to prevent seizure spread when all neuronal circuits in the brain are maximally active. These seizures are highly reproducible and are electrophysiologically consistent with human seizures (White, H.S., A.S. Bender, and E.A. Swinyard, Effect of the selective N-methyl-D-aspartate receptor agonist 3-(2-carboxypiperazin-4-yl)propyl-1-phosphonic acid on [3H]flunitrazepam binding. Eur J Pharmacol, 1988. 147(1): p. 149-51; Swinyard, E.A., Electrically induced convulsions, in Experimental Models of Epilepsy, D.B. Purpura, et al., Editors. 1972, Raven Press: New York. p. 443-58; Swinyard, E.A., Experimental Models of Epilepsy: A Manual for the Laboratory Worker. Electrically induced convulsions, ed. J.K.P. D.P Purpura, D. Tower, D.M. Woodbry, R. Walter. 1972, New York: Raven Press. 433-438. 5; Barton, M. E., et al., Pharmacological characterization of the 6 Hz psychomotor seizure model of partial epilepsy. Epilepsy Res, 2001. 47: p. 217-27). For all tests based on IVIES convulsions, 60 Hz of alternating current (50 mA in mice) is delivered for 0.2 s by corneal electrodes which have been primed with an electrolyte solution containing an anesthetic agent (0.5% tetracaine HCL). Mice are tested at various intervals following doses of 0.5, 1 and 3 mg/kg of anti-FGFR1c mAb R1MAb1 described in WO 2012/158704 given by i.p. injection weekly. These antibodies activate the FGF21 receptor. We observe that a number of the animals are protected from IVIES-induced seizures as evidenced by abolition of the hindlimb tonic extensor component of the seizure.

### Example 2. An Anti-FGFR1c Agonist Antibody Inhibits Seizures in the MES Model

The 6 Hz is a model that evaluates the ability of test agent to block psychomotor seizures induced by a low-frequency (6 Hz), long-duration (3 sec) stimulus delivered through corneal electrodes (Toman, J.E.P., G.M. Everett, and R.M. Richards, The search for new drugs against epilepsy. Texas Reports on Biology & Medicine, 1952. 10: p. 96-104; Swinyard, E.A., Electrically induced convulsions, in Experimental Models of Epilepsy, D.B. Purpura, et al., Editors. 1972, Raven Press: New York. p. 443-58; Swinyard, E.A., Experimental Models of Epilepsy: A Manual for the Laboratory Worker. Electrically induced convulsions, ed. J.K.P. D.P Purpura, D. Tower, D.M. Woodbry, R. Walter. 1972, New York: Raven Press. 433-438. 5; and Barton, M. E., et al., Pharmacological characterization of the 6 Hz psychomotor seizure model of partial epilepsy. Epilepsy Res, 2001. 47: p. 217-27).

Adult male CF1 mice (18-25 g) are pretreated intraperitoneally (i.p.) with 0.5, 1 and 3 mg/kg of anti-FGFR1c mAb R1MAb1. Each treatment group (n=4 mice/group) is examined for anti-convulsive effects at one of five time points (¼, ½, 1, 2, and 4 hr) after treatment with the test compound. Following pretreatment, each mouse receives a drop of 0.5% tetracaine hydrochloride applied to each eye. The mouse is then challenged with the low-frequency (6 Hz) stimulation for 3 sec delivered through corneal electrodes. The low-frequency, long-duration stimuli are initially delivered at 32 mA intensity. Animals are manually restrained and released immediately following the stimulation and observed for the presence or absence of seizure activity. Typically, the 6 Hz stimulation results in a seizure characterized by a minimal clonic phase that is followed by stereotyped, automatistic behaviors, including twitching of the vibrissae, and Straub-tail. We observe that a number of the animals did not display such behaviors and are considered protected.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. Use of an FGF21 receptor activator in the manufacture of a medicament for the treatment of epilepsy.

2. The use of claim 1, wherein the FGF21 receptor activator is selected from the group consisting of FGF21, an anti-FGFR1c antibody, an anti-KLB antibody, and a bispecific anti-FGFR1c/KLB antibody.

3. The use of claim 2, wherein the FGF21 receptor activator is FGF21.

4. The use of claim 3, wherein FGF21 is conjugated to a heterologous molecule.

5. The use of claim 4, wherein the heterologous molecule is PEG.

6. The use of claim 4, wherein the heterologous molecule is a polypeptide.

7. The use of claim 6, wherein the polypeptide is an antibody Fc.

8. The use of claim 7, wherein the antibody if IgG1.

9. The use of claim 2, wherein the FGF21 receptor activator is an anti-FGFR1c antibody.

10. The use of claim 9, wherein the anti-FGFR1c antibody binds to a peptide selected from the group consisting of KLHAVPAAKTVKFKCP (SEQ ID NO: 3) and FKPDHRIGGYKVRY (SEQ ID NO: 4).

11. The use of claim 2, wherein the FGF21 receptor activator is an anti-KLB antibody.

12. The use of claim 11, wherein the anti-KLB antibody is wherein the anti-KLB antibody is selected from the group consisting of 16H7 and h5h23.

13. The use of claim 2, wherein the FGF21 receptor activator is a bispecific anti-FGFR1c/KLB antibody.

14. The use of claim 13, wherein the bispecific anti-FGFR1c/KLB antibody binds to a KLB epitope within a fragment of KLB consisting of the amino acid sequence SSPTRLAVIPWGVRKLLRWVRRNYGDMDIYITAS (SEQ ID NO: 5).

15. The use of claim 14, wherein the bispecific anti-FGFR1c/KLB antibody comprises an anti-FGFR1c arm comprising amino acid sequences from YW182.5 YGDY and an anti-KLB arm comprising amino acid sequences from anti-8C5.K4.M4L.H3.KNV.

16. The use of claim 1, wherein the medicament is administered subcutaneously.

17. The use of claim 1, wherein the medicament is for administration with one or more additional therapeutics selected from the group consisting of: levetiracetam (“KEPPRA™”), Levetiracetam Extended Release (XR) (“KEPPRA XR™”), lamotrigine (“LAMICTAL™”), lamotrigine XR (“LAMICTAL XR™”), oxycarbazepine (“TRILEPTAL®”), carbamazepine (“TEGRETOL®”), lacosamide (“VIMPAT®”), valproic acid (“VPA”), and perampanel (“FYCOMPA®”).

18. A method of treating epilepsy in an individual comprising administering to the individual an effective amount of an FGF21 receptor activator.

19. The method of claim 18, wherein the FGF21 receptor activator is selected from the group consisting of FGF21, an anti-FGFR1c antibody, an anti-KLB antibody, and a bispecific anti-FGFR1c/KLB antibody.

20. The method of claim 19, wherein the FGF21 receptor activator is FGF21.

21. The method of claim 20, wherein FGF21 is conjugated to a heterologous molecule.

22. The method of claim 21, wherein the heterologous molecule is PEG.

23. The method of claim 21, wherein the heterologous molecule is a polypeptide.

24. The method of claim 23, wherein the polypeptide is an antibody Fc.

25. The method of claim 24, wherein the antibody if IgG1.

26. The method of claim 19, wherein the FGF21 receptor activator is an anti-FGFR1c antibody.

27. The method of claim 26, wherein the anti-FGFR1c antibody binds to a peptide selected from the group consisting of KLHAVPAAKTVKFKCP (SEQ ID NO: 3) and FKPDHRIGGYKVRY (SEQ ID NO: 4).

28. The method of claim 19, wherein the FGF21 receptor activator is an anti-KLB antibody.

29. The method of claim 28, wherein the anti-KLB antibody is selected from the group consisting of 16H7 and h5h23.

30. The method of claim 19, wherein the FGF21 receptor activator is a bispecific anti-FGFR1c/KLB antibody.

31. The method of claim 30, wherein the bispecific anti-FGFR1c/KLB antibody binds to a KLB epitope within a fragment of KLB consisting of the amino acid sequence SSPTRLAVIPWGVRKLLRWVRRNYGDMDIYITAS (SEQ ID NO: 5).

32. The method of claim 31, wherein the bispecific anti-FGFR1c/KLB antibody comprises an anti-FGFR1c arm comprising amino acid sequences from YW182.5 YGDY and an anti-KLB arm comprising amino acid sequences from anti-8C5.K4.M4L.H3.KNV.

33. The method of claim 18, wherein the FGF21 receptor activator is administered subcutaneously.

34. The use of claim 18, further comprising the administration of one or more additional therapeutics selected from the group consisting of: levetiracetam (“KEPPRA™”), Levetiracetam Extended Release (XR) (“KEPPRA XR™”), lamotrigine (“LAMICTAL™”), lamotrigine XR (“LAMICTAL XR™”), oxycarbazepine (“TRILEPTAL®”), carbamazepine (“TEGRETOL®”), lacosamide (“VIMPAT®”), valproic acid (“VPA”), and perampanel (“FYCOMPA®”).


SEQUENCE LISTING (5 sequences)

SEQ ID NO: 1 - protein, 820 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 2 - protein, 992 aa, Homo sapiens
FSGDGRAIWSKNPNFTPVNESQLFLYDTFPKNFFWGIGTGALQVEGSWKKDGKGPSIWDHFIHTHLKNVSSTNGSSDSYIFLEKDLSALDFIGVSFYQFSISWPRLFPDGIVTVANAKGLQYYSTLLDALVLRNIEPIVTLYHWDLPLALQEKYGGWKNDTIIDIFNDYATYCFQMFGDRVKYWITIHNPYLVAWHGYGTGMHAPGEKGNLAAVYTVGHNLIKAHSKVWHNYNTHFRPHQKGWLSITLGSHWIEPNRSENTMDIFKCQQSMVSVLGWFANPIHGDGDYPEGMRKKLFSVLPIFSEAEKHEMRGTADFFAFSFGPNNFKPLNTMAKMGQNVSLNLREALNWIKLEYNNPRILIAENGWFTDSRVKTEDTTAIYMMKNFLSQVLQAIRLDEIRVFGYTAWSLLDGFEWQDAYTIRRGLFYVDFNSKQKERKPKSSAHYYKQIIRENGFSLKESTPDVQGQFPCDFSWGVTESVLKPESVASSPQFSDPHLYVWNATGNRLLHRVEGVRLKTRPAQCTDFVNIKKQLEMLARMKVTHYRFALDWASVLPTGNLSAVNRQALRYYRCVVSEGLKLGISAMVTLYYPTHAHLGLPEPLLHADGWLNPSTAEAFQAYAGLCFQELGDLVKLWITINEPNRLSDIYNRSGNDTYGAAHNLLVAHALAWRLYDRQFRPSQRGAVSLSLHADWAEPANPYADSHWRAAERFLQFEIAWFAEPLFKTGDYPAAMREYIASKHRRGLSSSALPRLTEAERRLLKGTVDFCALNHFTTRFVMHEQLAGSRYDSDRDIQFLQDITRLSSPTRLAVIPWGVRKLLRWVRRNYGDMDIYITASGIDDQALEDDRLRKYYLGKYLQEVLKAYLIDKVRIKGYYAFKLAEEKSKPRFGFFTSDFKAKSSIQFYNKVISSRGFPFENSSSRCSQTQENTECTVCLFLVQKKPLIFLGCCFFSTLVLLLSIAIFQRQKRRKFWKAKNLQHIPLKKGKRVVS

SEQ ID NO: 3 - protein, 16 aa, Artificial Sequence
  Description of Artificial Sequence Synthetic peptide
KLHAVPAAKTVKFKCP

SEQ ID NO: 4 - protein, 14 aa, Artificial Sequence
  Description of Artificial Sequence Synthetic peptide
FKPDHRIGGYKVRY

SEQ ID NO: 5 - protein, 34 aa, Artificial Sequence
  Description of Artificial Sequence Synthetic polypeptide
SSPTRLAVIPWGVRKLLRWVRRNYGDMDIYITAS